(19) United States
(12) Patent Application Publication
(10) Pub. No.: US 2004/0116828 A1
(43) Pub. Date: Jun. 17, 2004

(54) Biological specimen collector and container with stand

(76) Inventor: Keith M. White JR., South Ogden, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/704,532

(22) Filed: Nov. 7, 2003

Related U.S. Application Data
(60) Provisional application No. 60/424,482, filed on Nov. 7, 2002.

(51) Int. Cl. B65D 81/00; A61B 5/00

(52) U.S. Cl. 600/573; 600/580; 221/33; 206/438; 422/104

(57) ABSTRACT

A specimen collection device is disclosed which is structured with an enlarged opening to facilitate the collection of bodily specimens, particularly the collection of urine from females. The specimen collection device generally comprises a collection bag, enlargement mechanism and closing member for sealing the collection bag after collection of the specimen. The specimen collection device further includes a port for accessing and removing the specimen from the collection bag. A storage device and packaging for dispensing the specimen collection device are also disclosed.

### CROSS-REFERENCE TO RELATED APPLICATION

This is a non-provisional application claiming priority to provisional application Serial No. 60/424,482 filed Nov. 7, 2002.

### BACKGROUND OF THE INVENTION

1. Field of the Invention

This invention relates to devices for collecting biological specimens, particularly urine samples, for medical use, and specifically relates to a collection container which is simple to dispense, use, handle and store after being filled with a biological specimen.

2. Description of Related Art

Biological specimen collection devices of the type used for collecting urine generally comprise bottles or tubes that have lids for closing the container. Occasionally, cups are used for collecting the specimen and then the specimen is transferred to a bottle or tube. Such specimen collection devices are generally suitable for use by males given male anatomical structure, but such collection devices are not very suitable for use by females. Thus, collection of a urine sample by females is usually messy and inconvenient when using known biological specimen collection devices.

Others have developed female urine collection devices which are structured to fit the female anatomy and reduce the likelihood of leaking, etc., but such device are not directed to structuring the device for containment of the biological specimen for later medical use or testing. Known devices are directed more to collection of urine for disposal in situations where the user is confined or bedridden.

Thus, it would be advantageous in the art to provide a biological specimen collection device that is structured for use by females which allows for the collection of a urine sample in a non-invasive, contained and clean manner. It would also be advantageous to provide a biological specimen collection device which is structured for facilitating the storage and retrieval of the specimen from the collection device, and to provide means for easily dispensing such collection devices in a specified place of use.

### BRIEF SUMMARY OF THE INVENTION

In accordance with the present invention, a biological specimen collection device is provided which is particularly structured for use by females in collecting a urine sample, and for doing so in a non-invasive and cleanly contained manner. The collection device is further structured to be securely closed for containment of the specimen within the collection device and to be easily transported, stored and handled for future accessing of the biological specimen. A dispensing device for the biological specimen collection device is also disclosed. A storage device for retaining one or more biological specimen devices is also disclosed. While the biological specimen collection device of the present invention is suitable for use in collecting urine samples of females, it is equally suited for use by men in collecting urine samples and can be used for the collection and retention of any other body fluid.

The biological specimen collector device of the present invention comprises a collection bag that is structured with an opening that is laterally adjustable to provide an enlarged opening that is conformable to the female anatomy, thereby facilitating the collection of a urine specimen in a clean and contained manner. The collection bag is generally characterized as comprising a receptacle that is closed on three sides to form an enclosure. The fourth side is structured as an opening that is configured with an enlarging mechanism which facilitates and retains the enlargement of the opening in a configuration suitable to conformance of the opening to the female anatomy.

The collection bag is also provided with a closing member that is structured to seal the open side of the collection bag after the specimen has been collected. The closing member preferably is formed with locking apparatus which assures a competent sealing of the collection bag.

The collection bag also comprises a means for retrieving the specimen from the collection bag. In particular, the collection bag may be structured with a closeable port that enables removal of the specimen from the collection bag through other than the open side. The port is preferably located at a low point of the collection bag to facilitate draining of the specimen. By providing a port, the collection bag can be sealed after collection of the specimen and does not need to be reopened to access the specimen, thereby making the device clean to use for medical staff.

The present invention further comprises a storage unit that is particularly structured to receive and retain at least one collection bag for storage. Preferably, the storage unit is capable of retaining or holding a plurality of collection bags. The storage unit is structured so that the retained collection bags are oriented with the port directed toward the user to thereby facilitate removal of the specimen from the collection bag. The storage unit is also configured to orient the collection bags in a manner which allows ready access to identifying information that may be printed on the outside of the collection bag.

The present invention also comprises packaging for dispensing of sterile collection bags from the packaging. The packaging device provides for easy access and removal of collection bags from the package and provides notification when the number of collection bags in the packaging is getting low.

### BRIEF DESCRIPTION OF THE SEVERAL VIEWS OF THE DRAWINGS

In the drawings, which depict what is currently considered to be the best mode for carrying out the invention:

FIG. 1 is a perspective view of a first embodiment of the present invention;

FIG. 2 is a plan view of the embodiment of the invention shown in FIG. 1;

FIG. 3 is a side view in elevation of the embodiment of the invention shown in FIG. 1;

FIG. 4 is a depiction in cross section of an exemplar closing member of the invention;

FIG. 5 is a perspective view of a second embodiment of the present invention;

FIG. 6 is a plan view of the embodiment of the invention shown in FIG. 5;

FIG. 7 is a side view in elevation of the embodiment of the invention shown in FIG. 5;

FIG. 8 is a perspective view of the storage device which is part of the invention;

FIG. 9 is a view in elevation of the storage device shown in FIG. 8;

FIG. 10 is an exploded perspective view of an exemplar port of the invention;

FIG. 11 is side view in elevation of the port illustrating the components in a closed position;

FIG. 12 is a view in cross section of the port shown in FIG. 11, taken at line 12-12;

FIG. 13 is a view in elevation of the front of a packaging device that is part of the invention;

FIG. 14 is a side view in elevation of the packaging device shown in FIG. 13;

FIG. 15 is a plan view of the packaging with the top side being closed; and

FIG. 16 is an alternative view in elevation of packaging of the invention.

### DETAILED DESCRIPTION OF THE INVENTION

The biological specimen collection device 10 of the present invention is shown in FIG. 1. The device 10 generally comprises a collection bag 12 having an access port 14 located near the bottom 16 of the collection bag 12 for accessing the specimen contained in the collection bag 12, and a closing member 18 located at the top 20 of the collection bag 12 for securing the upper edge 20 of the collection bag 12 in a closed and leakproof manner. The specimen collection device 10 further comprises an enlargement mechanism 22 for enlarging and retaining the upper edge 20 of the collection bag 12 in an open position for filling, as explained further hereafter.

The collection bag 12 may be made of any suitable material such as impermeable and leak-proof cloth or plastic, but is preferably made of a thicker gauge plastic material that is durable and transparent or semi-transparent. The collection bag 12 is closed about the sides and bottom 16 of the collection bag 12, but the upper edge 20 or side of the collection bag 12 provides an opening 24 through which the biological specimen may be received. The collection bag 12 may be imprinted with certain indicia 26, such as capacity indicators, and/or may be imprinted with certain information necessary for its use, such as notations of government regulations 28 and the patient's personal information 30.

The enlargement mechanism 22 of the collection bag 12 is structured to provide rigidity or semi-rigidity to the upper edge 20 of the collection bag 12 so that the opening 24 provided in the collection bag 12 is enlarged and remains in a stably open position during filling. The enlargement mechanism 22 can be structured in any number of ways to provide a selected shape and desired rigidity to the upper edge 20 of the collection bag 12, thereby assuring a competent fit of the collection device 10 to the user's body during collection of the specimen.

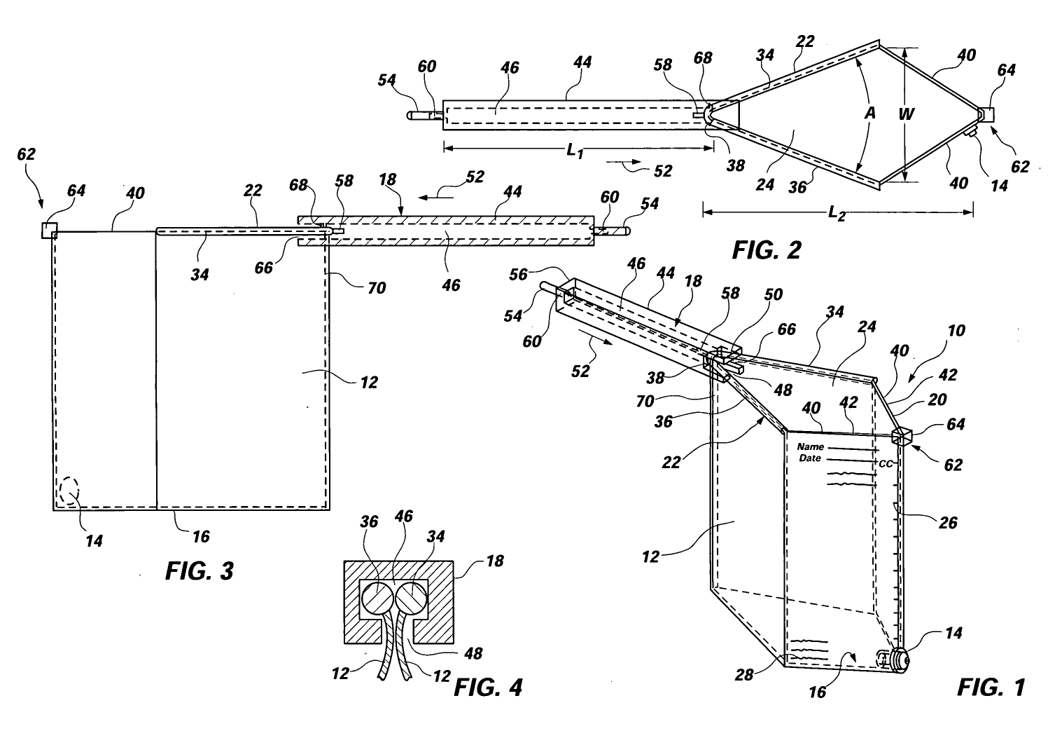

By way of example only, FIGS. 1 and 2 illustrate an exemplar means of carrying out the enlargement mechanism 22 of the invention. In general, the enlargement mechanism 22 is structured to be laterally expandable to enlarge the opening 24 of the collection bag 12. It is preferable, therefore, that the enlargement mechanism 22 be structured with an expansion or spring-loaded-type capacity to laterally expand and contract again. To that end, the exemplar enlargement mechanism 22 illustrated in FIGS. 1 and 2 comprises an angled piece of flexible plastic or metal having a first leg 34 and a second leg 36 joined together by a flexible connector or elbow 38. By virtue of the flexibility of the material used in the enlargement mechanism 22, the first leg 34 and second leg 36 are able to flexibly move toward and away from each other to facilitate enlargement of the opening 24 of the collection bag 12. The enlargement mechanism 22 may be structured in any other suitable manner, for example, comprising a first and second leg joined by a flexible connector comprising a spring-loaded mechanism, such as a flexible metal pin or a spring-loaded pivot.

As shown further in FIGS. 1 and 3, the top or upper edge 20 of the collection bag 12 is attached, at least in part, to the first leg 34 and second leg 36 of the enlargement mechanism 22. Exemplar means of attaching the collection bag 12 to the enlargement mechanism 22 may include bonding or welding the upper edge 20 of the bag 12 to the first leg 34 and second leg 34, or forming a sleeve at the upper edge 20 of the bag 12 and inserting the respective first and second legs 34, 36 into the sleeves or by forming the first leg 34 and second leg 36 with a downwardly extending channel into which the top edge 20 of the bag 12 is received and secured. Numerous other methods known to those of skill in the art may be employed to securely attach the bag 12 to the enlargement mechanism 22.

As shown in FIGS. 1-3, the upper edge 20 of the collection bag 12 is only partially attached to the enlargement mechanism 22. That is, a portion 40 of the upper edge 20 is not attached to the enlargement mechanism 22 and, consequently, the opening 24 is able to laterally expand. The expansion mechanism 22 may be selectively structured or made from a particularly selected material to provide a desired angle A (FIG. 2) of opening and a desired laterally opening width W. The portion 40 of the upper edge 20 that is not attached to the enlargement mechanism 22 may also be structured with a stiffening means 42 to facilitate the retention of the opening 24 in an enlarged state during use.

It should be noted that the enlargement mechanism 22 may be structured in such a manner that the entire upper edge 20 of the collection bag 12 may be attached to the enlargement mechanism 22 and be laterally expanded to provide an opening 24 sufficient for placement against the body during collection of the specimen. For example, the enlargement mechanism 22 may be comprised of a flexible material that is oval shaped to provide lateral expansion of the upper edge 20 to also assume an oval shape.

The closing member 18 of the invention is generally structured to facilitate the closure of the opening 24 of the collection bag 12 to prevent the specimen from spilling or leaking out of the opening 24. The closing member 18 may be any suitable device or structure that accomplishes that objective. By way of example only, FIGS. 1-3 illustrate one exemplar device that may be used as a closing member 18 of the invention. As shown, the closing member 18 comprises an elongated bar 44 having an internally disposed channel 46 that extends a length L₁ (FIG. 2), which is slightly less than the overall length of the elongated bar 44. The length L₁ of the channel 46 is substantially equal to the length L₂ of the collection bag 12, as shown in FIG. 2.

As shown most clearly in FIG. 4, the channel 46 in the elongated bar 44 is further structured with a slot 48 which extends substantially the length of the channel 46 and extends to a first end 50 of the elongated bar 44, as best seen in FIG. 1. When the collection bag 12 has been filled with a specimen, the elongated bar 44 is slid in the direction of arrow 52 toward the collection bag 12. The force of the elongated bar 44 sliding along the enlargement mechanism 22 causes the first leg 34 and second leg 36 to come together so that they eventually are oriented parallel to each other.

The first leg 34 and second leg 36 move into and down the length of the channel 44 which further causes the first and second legs 34, 36 to be brought in close proximity to each other to seal the top of the collection bag 12, as shown in FIG. 4. The collection bag 12, near its attachment to the enlargement mechanism 22, slides through the slot 48 as the elongated bar 44 is slid along the length L₂ of the collection bag 12. The elongated bar 44 may be structured with a gripping member 54, positioned at or near the second end 56 of the elongated bar 44, to aid in sliding the closing member 18 back and forth along the upper edge 20 of the collection bag 12 when opening and closing the bag 12.

The closing member 18 is preferably structured with locking apparatus that aids in assuring that the collection bag 12 will remain closed. By way of example only, the enlargement mechanism 22 of the embodiment illustrated in FIGS. 1-3 is structured with a locking pin 58 that extends from the elbow 38 of the enlargement mechanism. Likewise, a locking pin receiving slot 60 is formed in the gripping member 54 which is sized to receive the locking pin 58 when the elongated bar 44 is slid entirely along the length L₂ of the collection bag 12 to seal the bag 12 closed. The locking pin 58 is held in the locking pin receiving slot 60 by friction fit.

The collection bag 12 may also be provided with a second locking mechanism 62 which, in the embodiment illustrated in FIGS. 1-3, comprises a tab 64 shaped to be snuggly received in the open end 66 of the channel 44 when the elongated bar 44 is slid entirely over the length L₂ of the collection bag 12. The tab 64 is secured in the open end 66 of the elongated bar 44 by friction fit.

To use the specimen collection device 10, the collection bag 12 is preferably closed, with the closing member 18 extended entirely along the length L₂ of the collection bag 12, to assure sterile conditions within the interior of the collection bag 12. The user grips the gripping member 54 at the second end 56 of the closing member 18 and grips the tab 64 at the first end 50 of the closing member 18 and pulls in opposing directions. In so doing, the closing member 18 slides along the upper edge 20 of the collection bag 12 in a direction opposite arrow 52. As the closing member 18 is slid in that direction, the enlargement mechanism 22 slides out of the channel 46 of the closing member 18 and the elbow 38 begins to force the first leg 34 apart from the second leg 36 to begin enlargement of the opening 24 of the collection bag 12.

A stopping device 68 is preferably positioned near the open end 66 of the elongated bar 44 to prevent the enlargement mechanism 22 from being pulled entirely out of the channel 46 of the elongated bar 44. The stopping device 68 may, for example, be a pin that extends downwardly from the channel 46 of the elongated bar 44 to contact the elbow 38 of the enlargement mechanism 22 and prevent its further movement through the channel 46. Other stopping devices may be equally suitable.

When the opening 24 is laterally expanded to a desired width W, the upper edge 20 of the collection bag 12 is positioned against the body, surrounding the urethra, and is held in place by grasping the closing member 18. When the collection bag 12 has been filled with the specimen, the upper edge 20 of the collection bag 12 is moved away from the body, the user grasps the closing member 18 near the first end 50, holds the bag 12 approximately at a point 70 below the elbow 38 of the enlargement mechanism 22 and slides the closing member 18 in the direction of arrow 52 until the closing member 18 has been moved across the entire length L₂ of the collection bag 12. The user assures closure of the collection bag 12 by sliding the closing member 18 until the locking pin 58 is engaged within the locking pin receiving slot 60, and the tab 64 is lockingly engaged in the open end 66 of the closing member 18.

In the embodiment shown in FIGS. 1-3, the shape of the collection bag 12 is shown as having a quadrilateral geometry in lateral cross section, as best seen by FIG. 2. However, the illustrated shape or geometry of the collection bag 12 is merely illustrative and is not intended to convey a preferred or necessary shape or dimension of the collection bag 12. Many shapes and dimensions of the collection bag are possible. It may be a desired objective to configure the collection bag 12 with a flattened bottom 16 as illustrated in FIG. 1 to provide the collection bag 12 with an ability to be somewhat free-standing. It should also be noted that in the embodiment of FIGS. 1-3, the length L₁ of the closing member 18 is substantially the same as the length L₂ of the collection bag 12.

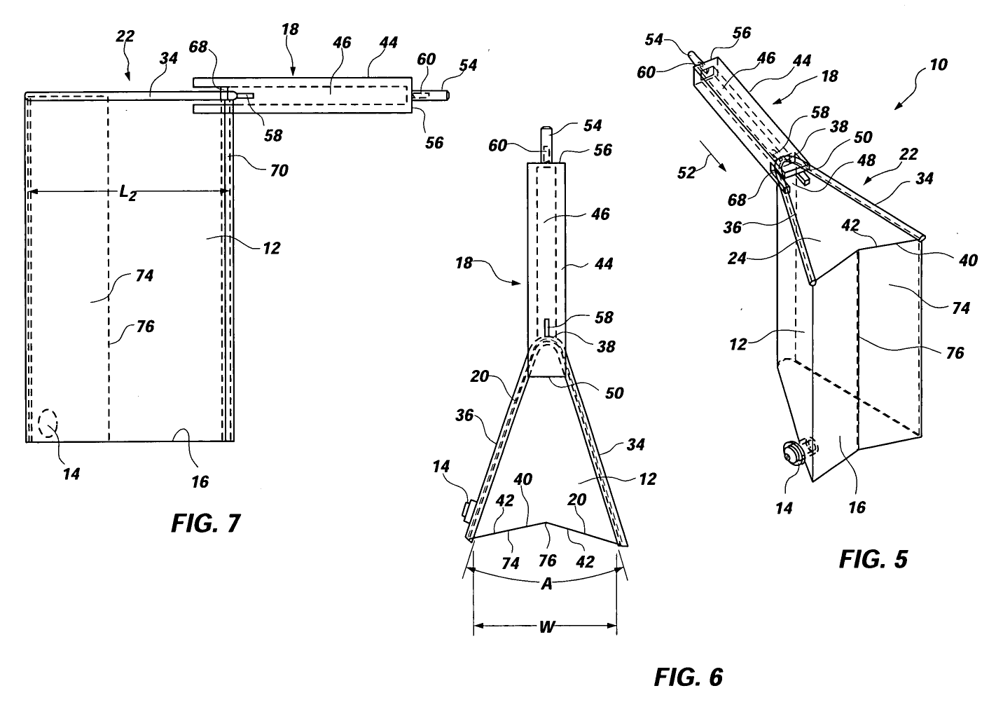

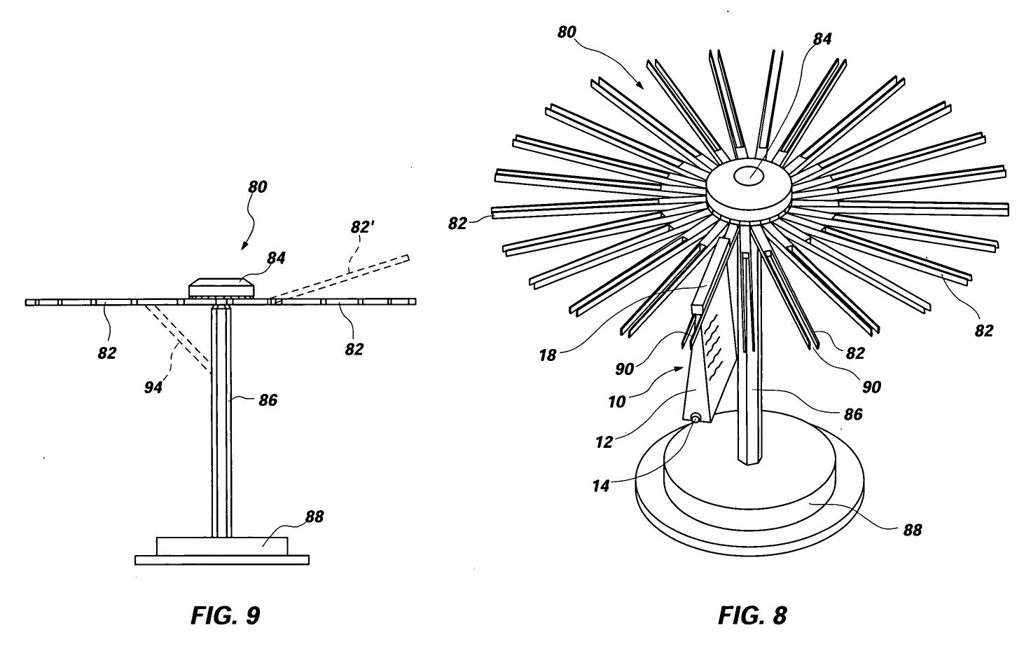

In an alternative embodiment, shown in FIGS. 5-7, the footprint of the collection bag 12 may be made smaller by structuring the collection bag 12 with an inwardly folding pleat 74. Like features of the embodiment shown in FIGS. 5-7 are designated with like reference numerals to those features shown in the prior embodiment illustrated in FIGS. 1-4. It can be seen that the inwardly folding pleat 74 enables the length L₂ (FIG. 7) of the collection bag 12 to be of smaller dimension than the previous embodiment. As a result, the collection bag 12 of this embodiment may be easier to handle and to store.

The alternative embodiment of the invention shown in FIGS. 5-7 operates in a manner very similar to the prior embodiment. Specifically, the collection bag 12 is preferably in a closed position with the closing member 18 housing the enlargement mechanism 22 within the channel 46 of the elongated bar 44 to bring the first leg 34 and second leg 36 of the enlargement mechanism 22 in close proximity to one another, thereby sealing the collection bag 12 and maintaining the sterile conditions of the interior. In use, the closing member 18 is moved in a direction opposite arrow 52 to slide the enlargement mechanism 22 out of the channel 46 of the elongated bar 44. In doing so, the elbow 38 of the enlargement mechanism 22 causes the first leg 34 and second leg 36 to move apart from each other to laterally enlarge the opening 24 of the collection bag 12.

In this embodiment, the pleat 74 is initially folded inwardly, along fold line 76, toward the interior of the collection bag 12, as illustrated in FIG. 7. As the enlargement mechanism 22 reaches a selected angle A and the selected width W of the opening is achieved, the pleat 74 begins to fold outwardly as shown in FIGS. 5 and 6. A generally triangular-shaped opening 24, as viewed in lateral cross section, results. The upper edge 20 of the collection bag 12 is positioned against the body and may be held in place by grasping the closing member 18.

After the specimen has been collected in the collection bag 12, the upper edge 20 of the collection bag 12 is move away from the body. The collection bag 12 is then sealed by sliding the closing member 18 in the direction of arrow 52 to slide the closing member 18 over the enlargement mechanism 22. Prior to completely moving the closing member 18 over the upper edge 20 of the bag 12, the fold line 76 of the pleat 74 is tapped with a finger to urge the pleat 74 back inwardly toward the interior of the bag 12. As the first leg 34 and second leg 36 come together in adjoining proximity, the portion 40 of the upper edge 20 which is not attached to the enlargement mechanism 22 is caught between the first leg 34 and second leg 36 and is sealed therebetween when the closing member 18 is positioned fully along the length L₂ of the collection bag 12. Stiffening means 42 may be associated with the portion 40 of the upper edge 20 which is not attached to the enlargement mechanism 22.

The embodiment of the collection device 10 shown in FIGS. 5-7 is also structured with locking apparatus to assure the closure of the collection bag 12. Again, the enlargement mechanism 22 may be structured with a locking pin 58 which is slidingly received into a corresponding locking pin receiving slot 60 located within the gripping member 54 of the closing member 18. The locking pin 58 is held in place within the locking pin receiving slot 60 by friction fit. As noted before, any number and variety of locking mechanisms may be employed in the closing member and/or the collection bag 12 to assure a competent sealing of the opening 24 of the collection bag 12.

Once a specimen has been collected in the collection bag 12, it may be retained on a storage device that also comprises an aspect of the invention. As shown in FIGS. 8 and 9, the storage device 80 of the present invention is particularly structured to receive and retain one or more specimen collection devices 10. The storage device 80 is configured to retain the specimen collection devices 10 in an orientation which gives ready access to the specimen contained in the collection bag 12 and access to the information printed on the collection bag 12.

The storage device 80 generally comprises a plurality of arms 82 that radiate from a central point of the storage device 80, thereby allowing the storage device 80 to hold many collection bags 12 in a small amount of space. The storage device 80 may preferably comprise a rotatable hub 84 from which the arms 82 radiate. The hub 84 may be pivotally or rotationally attached to an upright post 86 that is, in turn, connected to or integrally formed with a base 88.

Each of the arms 82 is particularly structured to receive and retain a specimen collection device 10. By way of example, each arm 82 may be structured with an elongated slot 90 which is sized in width to receive a portion of the collection bag 12 and/or closing member 18. As shown, the slot 90 may receive the portion of the collection bag 12 immediately below the closing member 18 while the closing member 18 rests above the slot 90. Other equally suitable means of structuring the arms 82 to accommodate retention of the collection devices 10 thereon are possible. It is only important that the structure or configuration be such that the collection bag 12 is oriented so that the port 14 of the collection bag 12 is easily accessible.

The arms 82 shown in FIGS. 8 and 9 are radiating outwardly in a substantially horizontal plane from the hub 84. However, as illustrated more fully in FIG. 9, it may be desirable to provide cantilevered supports 94 (shown in phantom) extending from the arms 82 of the storage device 80 to the upright post 86. Such cantilevered supports 94 may be useful or necessary when associating heavier weight collection bags 12 with the storage device 80. If cantilevered supports 94 are employed in the invention, the upright post would obviously rotate relative to the base 88 rather than the hub 84 rotating relative to the upright post 86. Alternatively, as suggested in phantom in FIG. 9, the arms 82′, may be oriented at an angle to the upright post 86 to provide further support and strength to the arms 82′. It may be necessary to modify the structure or dimension of the collection device 10 accordingly to more suitably accommodate the hanging of the bag 12 on an upwardly angled arm 82′.

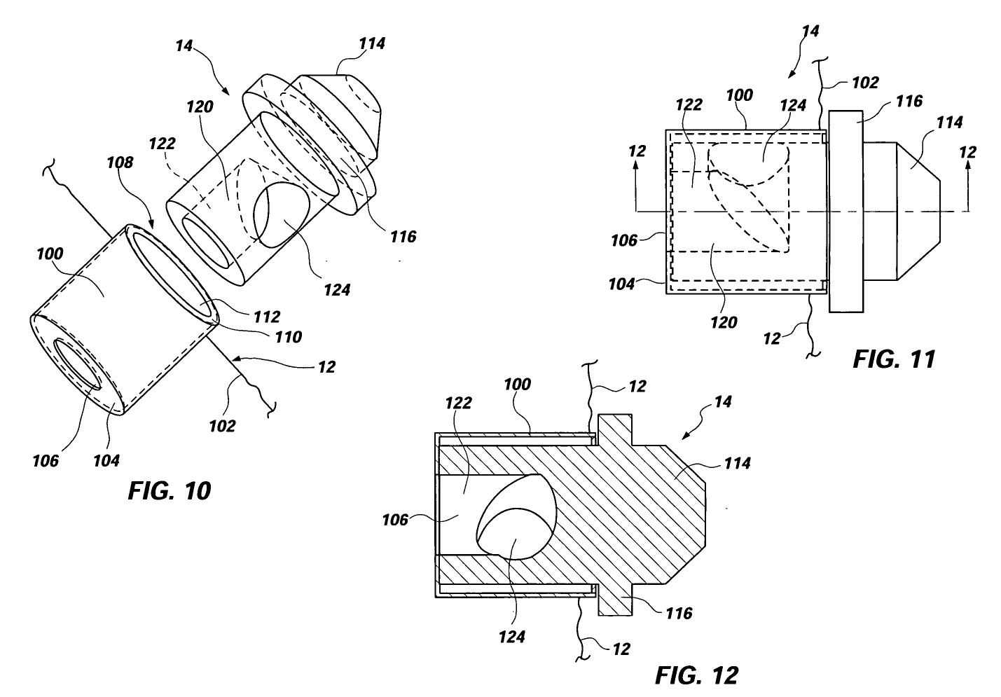

Turning to the accessing of the specimen from the specimen collection device 10, each collection bag 12 is structured with an access port 14 that allows removal of the specimen from the collection bag 12. FIGS. 10-12 illustrate, by way of example only, an exemplar type of access port 14 that may be used with the specimen collection device 10. The port 14, in this embodiment, comprises a sleeve 100 that is sealingly secured to and passes through the wall 102 of the collection bag 12. The sleeve 100 has a first end 104 which is oriented toward the interior of the collection bag 12. The first end 104 may be substantially open or, as illustrated, may be partially closed leaving an opening inner 106 of defined diameter. The sleeve 100 has a second end 108 which is oriented away from the wall 102 of the collection bag 12. The second end 108 is formed with an inwardly extending wall 110 which provides an opening 112.

A cylindrical hollow plug 114 is sized in circumference to be fittingly received through the opening 112 of the sleeve 100 and to move longitudinally within the sleeve 100. The plug 114 is fitted with a stop collar 116 which provides means by which the plug 114 may be grasped for pulling outward from the sleeve 100 and which provides a stop to prevent the plug 114 from moving too far within the sleeve 100. The plug 114 is structured with a fluid channel 120 to provide egress for the specimen material from within the bag 12. The fluid channel 120 may preferably comprise a first channel portion 122 which extends axially through the plug 114 and a second channel portion 124 which connects to and extends radially from the first channel portion 122.

When the plug 114 is positioned within the sleeve 100, as illustrated in FIGS. 11 and 12, the stop collar 116 registers against the wall 110 of the sleeve 100 and the tight tolerance between the plug 114 and sleeve 100 prevent any fluid from escaping from the port 14. When plug 114 is pulled outwardly from the sleeve 100 until the second channel portion 124 of the fluid channel 120 extends just beyond the wall 110 of the sleeve 100, specimen material from within the bag 12 is allowed to flow through the opening 106 in the sleeve 100 and into the fluid channel 120 from which it exits through the second portion 124 of the fluid channel 120. Other suitable means may be employed as an access port through the wall 102 of the specimen collection device to drain the specimen from the collection bag 12.

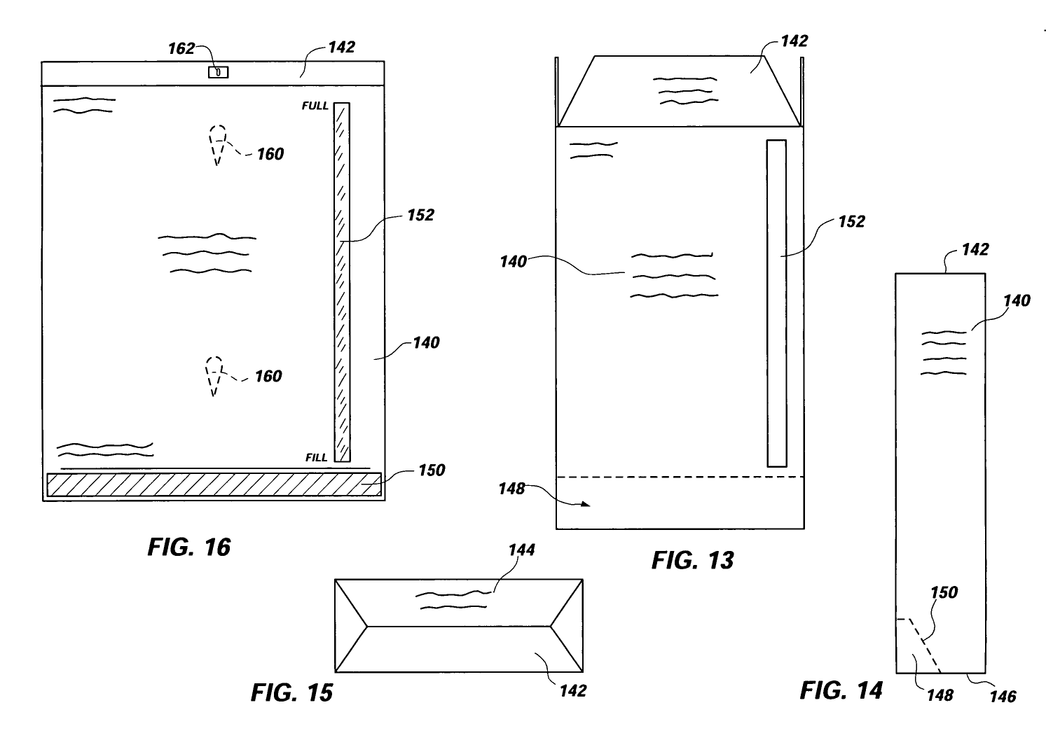

The present invention further comprises an exemplar means of providing packaging for a plurality of biological specimen collection devices to permit the dispensing of the collection device in a venue where the collection devices will be used. As shown in FIG. 13, the packaging may comprise an outer box 140 having a first openable end 142 for inserting a plurality of biological specimen collection devices into the box 140. The first openable end 142 is illustrated in FIG. 15 in a closed position, and suggests that written indicia 144 may be contained on the packaging. The box 140 also has a substantially closed end 146, shown in FIG. 14. A perforated section 148 near the closed end 146 of the box 140 is removable from the box 140 to provide an opening 150 through which a biological specimen collection device may be retrieved. The box 140 may be structured with a transparent window 152 through which one may observe when the number of devices in the box 140 is getting low or when the box 140 is empty.

FIG. 16 illustrates that the box 140 may be secured to a wall by wall brackets 160 (shown in phantom) and that the first openable end 142 of the box 140 may be fitted with a key lock 162 to secure the box 140 after filling to prevent tampering with the devices in the box 140.

The specimen collection device of the present invention is directed to use by females in collecting urine samples in a clean and contained manner. However, the specimen collection device may be used by males with equal success. Moreover, the specimen collection device may be used for collecting other specimens, such as blood or saliva. It will be clear to one of skill in the art that the specimen collection device of the present invention may be modified accordingly to enable use of the specimen collection device for various uses and various users. Hence, reference herein to specific configurations of the specimen collection device, storage device and packaging device are by way of example and not meant to limit the scope of the invention as recited in the claims.

What is claimed is:

1. A biological specimen collection device comprising:
a collection bag for retaining a biological specimen, said collection bag having an open side;
a closing member positioned to close said open side of said collection bag; and
an enlargement mechanism positioned near said open side of said collection bag to enlarge said open side for collection of a specimen.

2. The biological specimen collection device of claim 1 further comprising a closeable port connected to said collection bag for accessing and removing a specimen retained in said collection bag.

3. The biological specimen collection device of claim 1 further comprising locking apparatus for securing said collection bag to said closing member when said closing member is positioned to close said open side of said collection bag.

4. The biological specimen collection device of claim 1 further comprising a grasping member connected to said closing member for moving said closing member relative to said collection bag for opening and closing said open side.

5. The biological specimen collection device of claim 4 further comprising locking apparatus to secure said collection bag to said closing member when in a closed position.

6. The biological specimen collection device of claim 5 wherein said locking apparatus is associated with said grasping member.

7. The biological specimen collection device of claim 1 wherein said enlargement mechanism is comprised of a flexible material and comprises a first leg and a second leg connection by a flexible connector that imparts lateral expansion to said first leg and said second leg.

8. The biological specimen collection device of claim 7 wherein said enlargement mechanism is slidably received within said closing member.

9. The biological specimen collection device of claim 8 further comprising a locking mechanism positioned between said closing member and said enlargement mechanism to secure said enlargement mechanism to said closing member for sealing of said open side of said collection bag.

10. The biological specimen collection device of claim 1 wherein said collection bag is formed with a flattened bottom.

11. The biological specimen collection device of claim 10 wherein said collection bag has an interior space and is further formed with a pleat that is inwardly extendable toward said interior space.

12. The biological specimen collection device of claim 3 further comprising a second locking mechanism for securing said collection bag to said closing member, said second locking mechanism being spaced from said closing member when said collection bag is in an open and enlarged position.

13. The biological specimen collection device of claim 1 wherein said collection bag has an upper edge about said open side and said enlargement mechanism is secured to a portion of said upper edge, said collection bag further having stiffening means positioned along that portion of said upper edge which is not secured to said enlargement mechanism.

14. A storage device for retaining specimen collection devices, comprising:
an upright post having a hub;
a base for supporting said upright post;
at least one radially extending arm connected to said hub and positioned to rotate, said at least one arm being structured to receive and retain a specimen collection device comprising a collection bag, enlargement mechanism, closing member and port such that said port is readily accessible for removal of a specimen from said collection bag.

15. The storage device of claim 14 further comprising a plurality of radially extending arms, each arm being structured with a slot sized to receive and retain a portion of a specimen collection device therein.

16. The storage device of claim 14 wherein said at least one arm is supported by a cantilevered arm.

17. A packaging device for containing and dispensing specimen collection devices, comprising:
a box having at least six sides, said box being sized to retain and dispense a plurality of biological specimen collection bags;
an openable side for inserting biological specimen collection bags within said box;
at least one closed side having a perforated removable portion to provide an access opening through said box to remove biological specimen collection bags from within said box; and
a transparent window for viewing the contents of said box.

18. The packaging device of claim 17 further comprising a locking mechanism connected to said openable side.

19. The packaging device of claim 17 further comprising apparatus for attaching said box to a wall.
